(19) United States
(12) Patent Application Publication
(10) Pub. No.: US 2025/0262189 A1
(43) Pub. Date: Aug. 21, 2025

(54) TREATMENT OF DISEASES ASSOCIATED WITH REDUCTIVE STRESS

(71) Applicants: Taro INABA, Seattle, WA (US); Satoshi GOJO, Seattle, WA (US); RDiscovery, LLC, Seattle, WA (US); KYOTO PREFECTURAL PUBLIC UNIVERSITY CORPORATION, Kyoto-shi, Kyoto (JP)

(72) Inventors: Taro INABA, Tokyo (JP); Satoshi GOJO, Kyoto-fu (JP)

(21) Appl. No.: 18/858,493

(22) PCT Filed: Apr. 21, 2023
(86) PCT No.: PCT/US2023/019378

§ 371 Date: Oct. 21, 2024

Related U.S. Application Data
(60) Provisional application No. 63/333,800, filed on Apr. 22, 2022.

(51) Int. Cl. A61K 31/4184

(52) U.S. Cl. CPC A61K 31/4184

(57) ABSTRACT

Methods for reducing plasma GDF-15 in a subject, methods for reducing reductive stress in a subject, and methods for treating a subject having or at risk of developing a disease or dysfunction associated with reductive stress in a subject with ZLN-005 and other compounds of Formula (I), and salts, hydrates, deuterated analogs, and fluorinated analogs thereof.

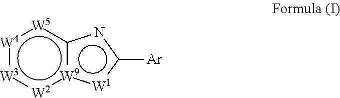

### 1. CROSS-REFERENCE TO RELATED APPLICATIONS

This application claims the priority benefit of U.S. provisional application No. 63/333,800, filed Apr. 22, 2022, the contents of which are incorporated herein in their entireties by reference thereto.

### 2. BACKGROUND

Oxidation and reduction (redox) reactions are critical to cellular energy generation and synthesis of cellular components. The cellular redox couples nicotinamide adenine dinucleotide (NAD+)/reduced NAD+ (NADH), phosphorylated NAD+ (NADP+)/reduced NADP+ (NADPH), and reduced glutathione (GSH)/GSH disulfide (GSSG) are responsible for the majority of cellular electron transfer. Imbalance of these redox couples in favor of the reduced forms (e.g., NADH) can cause reductive stress, which is associated with various diseases and disorders, for example mitochondrial, cardiovascular, liver, kidney, and lung diseases and dysfunctions, and cancer (see, e.g., Sharma et al., 2021, J Clin Invest. 131 (2): e136055; Chun et al., 2021, Cells 10:758; Perez-Torres et al., 2017, Int. J. Mol. Sci. 18:2098; Touyz et al., 2016, Circ Res. 119:969-971; Favia and Atlante, 2021, Int. J. Mol. Sci. 22:967; Xiao and Loscalzo, 2020, Antioxidants & Redox Signaling 32:18:1330-1347). Biomarkers of reductive stress include elevated growth/differentiation factor 15 (GDF-15), elevated high NADH/NAD+ ratios, and elevated lactate/pyruvate ratios.

New treatments for reducing reductive stress and for treating diseases and disorders associated with reductive stress are needed.

### 3. SUMMARY

The present disclosure is based, in part, on the discovery that 2-(4-tert-butylphenyl)-1H-benzimidazole (ZLN-005), an activator of Ppargc1a (PGC-1a) expression, decreases expression of reductive stress biomarker GDF-15 in an in vitro model of reductive stress. ZLN-005 has the following structure:

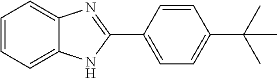 [SMILES: CC(C)(C)c1ccc(-c2nc3ccccc3[nH]2)cc1]

Accordingly, in one aspect, the disclosure provides methods for reducing plasma GDF-15 in a subject comprising administering to the subject an amount of an agent which is ZLN-005 or other compound of Formula (I):

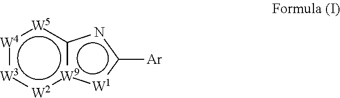

or a salt, hydrate, deuterated analog, or fluorinated analog thereof, in an amount effective to reduce the subject's plasma GDF-15 level, wherein:
Ar is

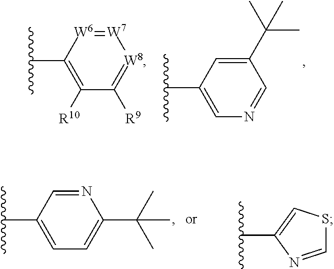

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁-4)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

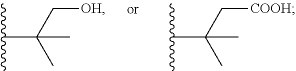

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

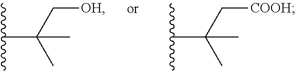

R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₅)alkoxycarbonyl, (C₁-C₆)alkoxycarbonylamino, methylthio, heterocyclyl, (C₁-C₁₀)oxaalkyl, CHR⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

In another aspect, the disclosure provides methods for reducing reductive stress in a subject in need thereof comprising administering to the subject an amount of an agent effective to reduce reductive stress in the subject, wherein the agent is a compound of Formula (I) (e.g., ZLN-005) or a salt, hydrate, deuterated analog, or fluorinated analog thereof.

In another aspect, the disclosure provides methods of treating a subject having or at risk of developing a disease or dysfunction associated with reductive stress comprising administering to the subject an amount of an agent effective to reduce reductive stress in the subject, wherein the agent is a compound of Formula (I) (e.g., ZLN-005) or a salt, hydrate, deuterated analog, or fluorinated analog thereof.

Further features compounds of Formula (I) and salts, hydrates, deuterated analogs, and fluorinated analogs thereof are described in Section 5.2 and numbered embodiments 1 to 3 and 80 to 93, infra.

Compounds of Formula (I) and salts, hydrates, deuterated analogs, and fluorinated analogs thereof can be administered in a pharmaceutical composition comprising the compound of Formula (I) or a salt, hydrate, deuterated analog, or fluorinated analog thereof. Exemplary features of pharmaceutical compositions are described in Section 5.3, infra.

Further features of the methods of the disclosure are described in Section 5.4 and numbered embodiments 1 to 99, infra.

### 4. BRIEF DESCRIPTION OF THE FIGURES

FIG. 1 shows the relative change in GDF-15 expression in HUVEC cells treated with azidothymidine (AZT) or AZT and ZLN-005 (AZT/ZLN) compared to non-treated control (Ctrl).

FIG. 2 shows the relative change in GDF-15 expression in HUVEC cells treated with azidothymidine (AZT) or AZT and ZLN-005 (AZT/ZLN) compared to non-treated control (Ctrl). P values are as follows: Ctrl vs. AZT=0.0002; Ctrl vs. AZT/ZLN=0.0002; AZT vs. AZT/ZLN=0.016.

### 5. DETAILED DESCRIPTION

This disclosure provides novel uses of ZLN-005 and other compounds of Formula (I) (as well as salts, hydrates, deuterated analogs, and fluorinated analogs thereof), for example in methods for reducing plasma GDF-15 and/or reducing reductive stress and/or treating a subject having or at risk of developing a disease or dysfunction associated with reductive stress. Exemplary compounds of Formula (I) and salts, hydrates, deuterated analogs, and fluorinated analogs thereof are described in Section 5.2. Exemplary pharmaceutical compositions comprising compounds of Formula (I) and salts, hydrates, deuterated analogs, and fluorinated analogs thereof are described in Section 5.3. Exemplary features of methods of the disclosure are described in Section 5.4.

### 5.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

The term elevated, with reference to a molecule (e.g., GDF-15, FGF-21, FGF-23, NADH, lactate) or ratio of molecules (e.g., NADH/NAD+, lactate/pyruvate), means that the amount of the molecule or the value of the ratio is higher than a reference amount or value. In some embodiments, the reference amount or value is the amount or value from a population of healthy subjects. In other embodiments, the reference amount or value is relative to a baseline for a given subject.

The term FGF-21 refers to fibroblast growth factor 21. Exemplary FGF-21 reference sequences are available in publicly available databases and include: UniProtKB accession no. Q9NSA1 and NCBI accession no. NP_061986.1. Those skilled in the art will appreciate that reference sequences sometimes include amino acid sequences for precursor proteins which are processed to provide mature protein.

The term FGF-23 refers to fibroblast growth factor 23. Exemplary FGF-23 reference sequences are available in publicly available databases and include: UniProtKB accession no. Q9GZV9 and NCBI accession no. NP_065689.1. Those skilled in the art will appreciate that reference sequences sometimes include amino acid sequences for precursor proteins which are processed to provide mature protein.

The term GDF-15 refers to growth/differentiation factor 15. Exemplary GDF-15 reference sequences are available in publicly available databases and include: UniProtKB accession no. Q99988 and NCBI accession no. NP_004855.2. Those skilled in the art will appreciate that reference sequences sometimes include amino acid sequences for precursor proteins which are processed to provide mature protein.

The term reductive stress refers to a situation in which a cell or population of cells (e.g., cells of a tissue or organ) has an excess accumulation of reducing equivalents (e.g., NADH, NADPH, or GSH), compared to a normal cell or population thereof. In some embodiments, reductive stress is NADH-reductive stress, in which there is an excess accumulation of NADH in a cell or population of cells compared to a normal cell or population of cells.

The term primary mitochondrial disease refers to a mitochondrial disease caused by a pathogenic mutation in mitochondrial or nuclear DNA encoding a protein involved in oxidative phosphorylation (oxphos). Examples of primary mitochondrial diseases include mitochondrial encephalomyopathy lactic acidosis and strokelike episodes (MELAS), Leigh syndrome, Kearns-Sayre syndrome, Alpers-Huttenlocher syndrome, and Ataxia neuropathy syndrome.

The term secondary mitochondrial dysfunction refers to mitochondrial dysfunction that is not caused by a pathogenic mutation in mitochondrial or nuclear DNA encoding a protein involved in oxidative phosphorylation. Secondary mitochondrial dysfunction can result from mutations in non-oxphos genes and can also be acquired from exposure to environmental factors and certain diseases and conditions. Mitochondrial dysfunction can be secondary to, for example, diabetes, heart disease, cancer, a kidney disease, a neurodegenerative disorder, Friedreich's Ataxia, Duchenne Muscular Dystrophy or Becker Muscular Dystrophy.

The terms treat, treating, treatment, and grammatical variations thereof as used herein, include reducing or ameliorating a disorder or dysfunction, and/or signs or symptoms associated therewith, or slowing or halting the progression thereof. It will be appreciated that, although not precluded, treating a disorder or dysfunction does not require that the disorder, dysfunction or symptoms associated therewith be completely eliminated. Treatments according to the disclosure may be applied prophylactically (e.g., to a subject at risk of developing a disease or dysfunction associated with reductive stress), palliatively or remedially. Prophylactic treatments can be administered to a subject prior to onset of a sign or symptom, during early onset of a sign or symptom (e.g., upon initial signs and symptoms), or after an established development of a sign or symptom. Prophylactic administration can occur for several days to years prior to the manifestation of a symptom.

### 5.2. Compounds of Formula (I)

The methods of the disclosure comprise administering to a subject an amount of an agent which is a compound of Formula (I):

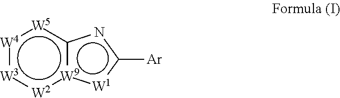

or a salt, hydrate, deuterated analog, or fluorinated analog thereof, wherein:
Ar is

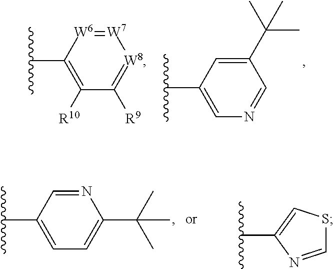

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁-4)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

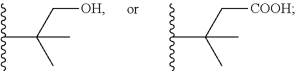

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

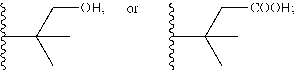

R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₅)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₅)alkyl.

Compounds of Formula (I) are further described in PCT publication no. WO 2021/262617, the contents of which are incorporated herein by reference in their entireties.

Exemplary compounds of Formula (I) include the following compounds:

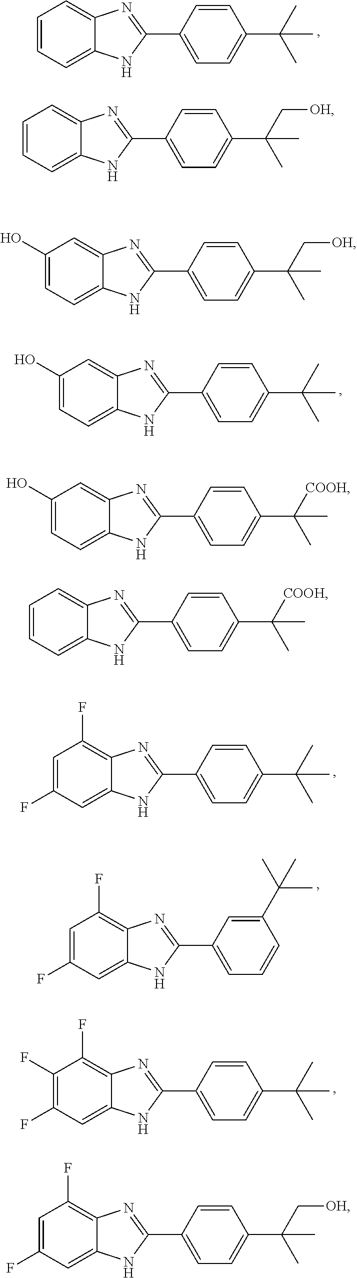

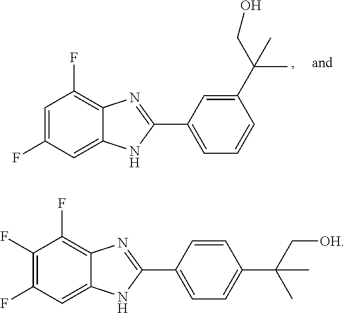 [SMILES: CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]

In some embodiments, the agent is

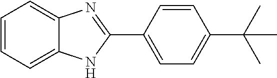 [SMILES: CC(C)(C)c1ccc(-c2nc3ccccc3[nH]2)cc1]

or a salt, hydrate, deuterated analog, or fluorinated analog thereof.

In some embodiments, the agent is

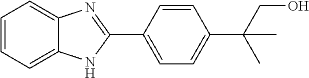 [SMILES: CC(C)(CO)c1ccc(-c2nc3ccccc3[nH]2)cc1]

or a salt, hydrate, deuterated analog, or fluorinated analog thereof.

In some embodiments, the agent is

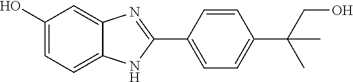 [SMILES: CC(C)(CO)c1ccc(-c2nc3cc(O)ccc3[nH]2)cc1]

or a salt, hydrate, deuterated analog, or fluorinated analog thereof.

In some embodiments, the agent is

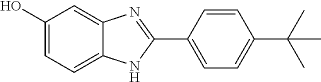 [SMILES: CC(C)(C)c1ccc(-c2nc3cc(O)ccc3[nH]2)cc1]

or a salt, hydrate, deuterated analog, or fluorinated analog thereof.

In some embodiments, the agent is

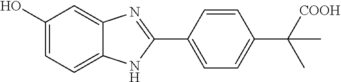 [SMILES: CC(C)(C(=O)O)c1ccc(-c2nc3cc(O)ccc3[nH]2)cc1]

or a salt, hydrate, deuterated analog, or fluorinated analog thereof.

In some embodiments, the agent is

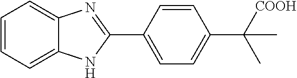 [SMILES: CC(C)(C(=O)O)c1ccc(-c2nc3ccccc3[nH]2)cc1]

or a salt, hydrate, deuterated analog, or fluorinated analog thereof.

In some embodiments, the agent is

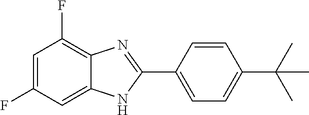 [SMILES: CC(C)(C)c1ccc(-c2nc3c(F)cc(F)cc3[nH]2)cc1]

or a salt, hydrate, deuterated analog, or fluorinated analog thereof.

In some embodiments, the agent is

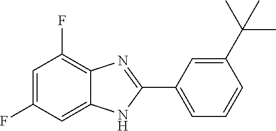 [SMILES: CC(C)(C)c1cccc(-c2nc3c(F)cc(F)cc3[nH]2)c1]

or a salt, hydrate, deuterated analog, or fluorinated analog thereof.

In some embodiments, the agent is

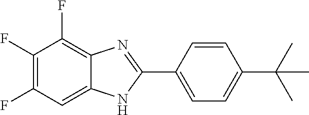 [SMILES: CC(C)(C)c1ccc(-c2nc3c(F)c(F)c(F)cc3[nH]2)cc1]

or a salt, hydrate, deuterated analog, or fluorinated analog thereof.

In some embodiments, the agent is

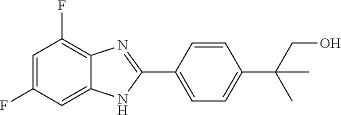 [SMILES: CC(C)(CO)c1ccc(-c2nc3c(F)cc(F)cc3[nH]2)cc1]

or a salt, hydrate, deuterated analog, or fluorinated analog thereof.

In some embodiments, the agent is

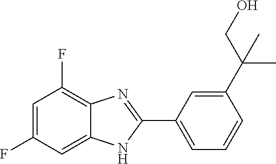 [SMILES: CC(C)(CO)c1cccc(-c2nc3c(F)cc(F)cc3[nH]2)c1]

or a salt, hydrate, deuterated analog, or fluorinated analog thereof.

In some embodiments, the agent is

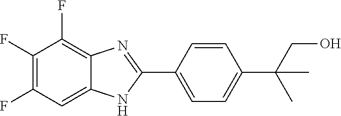 [SMILES: CC(C)(CO)c1ccc(-c2nc3c(F)c(F)c(F)cc3[nH]2)cc1]

or a salt, hydrate, deuterated analog, or fluorinated analog thereof.

In some embodiments, the agent is a compound of Formula (I) (e.g., one of the specific compounds of Formula (I) whose structure is shown in this Section) or a salt thereof.

In some embodiments, the agent is ZLN-005. In other embodiments, the agent is a salt of ZLN-005.

### 5.3. Pharmaceutical Compositions

Compounds of Formula (I) and salts, hydrates, deuterated analogs, and fluorinated analogs thereof can be formulated for the intended route of administration, for example according to techniques known in the art (e.g., as described in Allen et al., eds., 2012, Remington: The Science and Practice of Pharmacy, 22nd Edition, Pharmaceutical Press, London, UK). Suitable routes of administration include, but are not limited to, intravenous and oral routes of administration. Suitable routes also include pulmonary administration, including by inhalation. The most suitable route may depend upon the condition or disorder of the subject.

Compounds of Formula (I) and salts, hydrates, deuterated analogs, and fluorinated analogs thereof can be formulated as a pharmaceutical composition comprising a compound of Formula (I), or a salt, hydrate, deuterated analog, or fluorinated analog thereof and one or more pharmaceutical excipients, for example one or more excipients described in Handbook of Pharmaceutical Excipients, 8th Revised Ed. (2017), incorporated by reference in its entirety. The pharmaceutical compositions can be presented in unit dosage form.

### 5.4. Uses of Compounds of Formula (I)

The disclosure provides methods of treating subjects with compounds of Formula (I) (e.g., ZLN-005) and salts, hydrates, deuterated analogs, and fluorinated analog thereof. In the methods of the disclosure, the subjects are preferably mammals (e.g., primates or rodents such as mice or rats), most preferably humans.

In one aspect, the disclosure provides a method for reducing plasma GDF-15 in a subject comprising administering to the subject an amount of an agent effective to reduce the subject's plasma GDF-15, where the agent is a compound of Formula (I) (e.g., ZLN-005) or a salt, hydrate, deuterated analog, or fluorinated analog thereof.

In another aspect, the disclosure provides a method for reducing reductive stress in a subject in need thereof comprising administering to the subject an amount of an agent effective to reduce reductive stress in the subject, where the agent is a compound of Formula (I) (e.g., ZLN-005) or a salt, hydrate, deuterated analog, or fluorinated analog thereof. A reduction in reductive stress can be evidenced by, for example, a reduction in a biomarker associated with reductive stress following administration of the agent. In some embodiments, a reduction in reductive stress can be evidenced by a reduction in the NADH/NAD+ ratio in cells from a tissue or organ of the subject following administration of the agent, or a reduction in the subject's blood lactate/pyruvate ratio following administration of the agent.

In another aspect, the disclosure provides a method of treating a subject having or at risk of developing a disease or dysfunction associated with reductive stress comprising administering to the subject an amount of an agent effective to reduce reductive stress in the subject, wherein the agent is a compound of Formula (I) (e.g., ZLN-005) or a salt, hydrate, deuterated analog, or fluorinated analog thereof.

In the methods of the disclosure, the reductive stress can be NADH-reductive stress, which can be characterized by an excess accumulation of NADH in a cell or population of cells (e.g., a population of cells in a tissue or organ) compared to a normal cell or population of cells.

The methods of the disclosure can comprise administering an amount of the agent effective to reduce one or more biomarkers of reductive stress. Examples of biomarkers of reductive stress include plasma and tissue GDF-15, plasma and tissue FGF-21, plasma and tissue FGF-23, and blood lactate/pyruvate ratio. Biomarkers of reductive stress can be measured by methods known in the art, for example, by an enzyme-linked immunosorbent assay (ELISA). In some embodiments of the methods described herein, for a subject having a disease or dysfunction associated with reductive stress, treating can comprise reducing or alleviating one or more symptoms of the disease or dysfunction.

Diseases and dysfunctions associated with reductive stress can be associated with elevated plasma and/or tissue NADH/NAD+ ratios and/or elevated blood lactate/pyruvate ratio.

In some embodiments, the disease or dysfunction associated with reductive stress is a mitochondrial disease or dysfunction (e.g., primary mitochondrial disease or secondary mitochondrial dysfunction), a cardiovascular disease or dysfunction, a liver disease or dysfunction, a lung disease or dysfunction, a kidney disease or dysfunction or a cancer.

In some embodiments, the subject treated according to the methods of the disclosure has a primary mitochondrial disease, for example mitochondrial encephalomyopathy lactic acidosis and strokelike episodes (MELAS), Leigh syndrome, Kearns-Sayre syndrome, Alpers-Huttenlocher syndrome, or Ataxia neuropathy syndrome.

In other embodiments, the subject has a secondary mitochondrial dysfunction or is at risk of secondary mitochondrial dysfunction. For example, a subject can be at risk for mitochondrial dysfunction secondary to diabetes, heart disease, cancer, a kidney disease, or a neurodegenerative disorder. Additional examples of diseases that can put a subject at risk for secondary mitochondrial dysfunction include Friedreich's Ataxia, Duchenne Muscular Dystrophy, and Becker Muscular Dystrophy.

In some embodiments, the subject treated according to the methods of the disclosure has a cardiovascular disease or dysfunction (e.g., heart disease or atherosclerosis), or is at risk of developing a cardiovascular disease or dysfunction.

In some embodiments, the subject treated according to the methods of the disclosure has a liver disease or dysfunction (e.g., a chronic liver disease such as fatty liver disease or an acute liver disease such as acute liver failure) or is at risk of developing a liver disease or dysfunction.

In some embodiments, the subject treated according to the methods of the disclosure has a lung disease or dysfunction (e.g., a chronic lung disease such as cystic fibrosis or an acute lung disease such as acute lung injury) or is at risk of developing a lung disease or dysfunction.

In some embodiments, the subject treated according to the methods of the disclosure has a kidney disease or dysfunction (e.g., a chronic kidney disease or an acute kidney disease) or is at risk of developing a kidney disease or dysfunction.

In some embodiments, the subject treated according to the methods of the disclosure has a cancer (e.g., a solid tumor or hematological cancer) or is at risk of developing a cancer.

In some embodiments of the methods of the disclosure, the subject is receiving or has received treatment with one or more nucleoside analogs, for example azidothymidine (AZT). AZT is an antiretroviral medication used to treat HIV/AIDS. Accordingly, in some embodiments, the subject has HIV.

In some embodiments of the methods of the disclosure, the subject has an elevated plasma GDF-15 level. In some embodiments, the subject has a plasma GDF-15 level greater than 1000 ng/l, greater than 1100 ng/l, or greater than 1200 ng/l, for example as measured by an ELISA assay such as the Human GDF-15 Quantikine ELISA Kit (R&D Systems). In some embodiments, the methods of the disclosure comprise administering an amount of the agent effective to reduce the subject's plasma GDF-15 level (e.g., to a value within a normal range for healthy subjects).

In some embodiments of the methods of the disclosure, the subject has an elevated tissue GDF-15 level (e.g., measured in cells obtained from a biopsy). In some embodiments, the methods of the disclosure comprise administering an amount of the agent effective to reduce the subject's tissue GDF-15 level (e.g., to a value within a normal range for that tissue type in healthy subjects).

In some embodiments of the methods of the disclosure, the subject has an elevated plasma FGF-21 level. In some embodiments, the subject has a plasma FGF-21 level greater than 800 μg/ml, greater than 900 μg/ml, or greater than 1000 μg/ml, for example as measured by an ELISA assay such as the Human FGF-21 Quantikine ELISA Kit (R&D Systems). In some embodiments, the methods of the disclosure comprise administering an amount of the agent effective to reduce the subject's plasma FGF-21 level (e.g., to a value within a normal range for healthy subjects).

In some embodiments of the methods of the disclosure, the subject has an elevated tissue FGF-21 level (e.g., measured in cells obtained from a biopsy). In some embodiments, the methods of the disclosure comprise administering an amount of the agent effective to reduce the subject's tissue FGF-21 level (e.g., to a value within a normal range for that tissue type in healthy subjects).

In some embodiments of the methods of the disclosure, the subject has an elevated plasma FGF-23 level. In some embodiments, the subject has a plasma FGF-23 level greater than 200 RU/ml, greater than 300 RU/ml, or greater than 400 RU/ml, for example as measured by an ELISA assay such as the Quest Diagnostics test code 91931. In some embodiments, the methods of the disclosure comprise administering an amount of the agent effective to reduce the subject's plasma FGF-23 level (e.g., to a value within a normal range for healthy subjects).

In some embodiments of the methods of the disclosure, the subject has an elevated tissue FGF-23 level (e.g., measured in cells obtained from a biopsy). In some embodiments, the methods of the disclosure comprise administering an amount of the agent effective to reduce the subject's tissue FGF-23 level (e.g., to a value within a normal range for that tissue type in healthy subjects).

In some embodiments of the methods of the disclosure, the subject has an elevated blood lactate/pyruvate ratio. Respiratory chain defects usually result in lactate/pyruvate ratios of greater than 20, with lactate/pyruvate ratios greater than 30 suggestive of inherited disorders of the respiratory chain. In some embodiments, the subject has a blood lactate/pyruvate ratio of at least 20, at least 25, at least 30, or at least 35. Methods for measuring blood lactate/pyruvate ratios are commercially available, e.g., Quest Diagnostics test code 11296. In some embodiments, the methods of the disclosure comprise administering an amount of the agent effective to reduce the subject's lactate/pyruvate levels (e.g., to a value within a normal range for healthy subjects).

In some embodiments of the methods of the disclosure, the subject has an elevated NADH/NAD+ ratio in one or more cell types (e.g., measured in cells obtained from a biopsy). NADH/NAD+ ratios, can be measured, for example, using a commercially available kit, for example the Abcam NAD/NADH Assay Kit (Colorimetric) (catalog no. ab65348). In some embodiments, the methods of the disclosure comprise administering an amount of the agent effective to reduce the NADH/NAD+ ratio in one or more cell types (e.g., to a value within a normal range for that cell type in healthy subjects).

In some embodiments of the methods of the disclosure, the subject does not have systematic immune activation, for example cytokine release syndrome or sepsis.

In some embodiments of the methods of the disclosure, the subject does not have a neurodegenerative disorder.

In the methods of the disclosure, the agent can be administered by any suitable means, for example enterically. In some embodiments, the agent is administered by mouth.

In some embodiments of the methods of the disclosure, agent is administered in a dose ranging from 0.5 mg/kg to 1000 mg/kg per day. In some embodiments, the dose is 25 mg/kg to 1000 mg/kg per day.

### 6. EXAMPLES

### 6.1. Reduction of GDF-15 Expression by ZLN-005

A study was performed to evaluate the ability of ZLN-005 to mitigate the effects of reductive stress induced by azidothymidine (AZT), which is believed to inhibit mitochondrial respiratory chain complex I and II.

Human human umbilical vein endothelial (HUVEC) cells were cultured in HuMedia-EG2 medium (Kurabo) at 5×10⁴ cells per well in a six-well plate. Medium was supplemented with AZT (Sigma) at 5 μM, with or without ZLN-005 at 1 μM. Cells cultured without AZT and without ZLN-005 were used as non-treated control (NTC)). Medium was changed on days 2 and 4. Cells were harvested on Day 6 for analysis of GDF-15 mRNA expression by quantitative reverse transcription PCR (RT-qPCR).

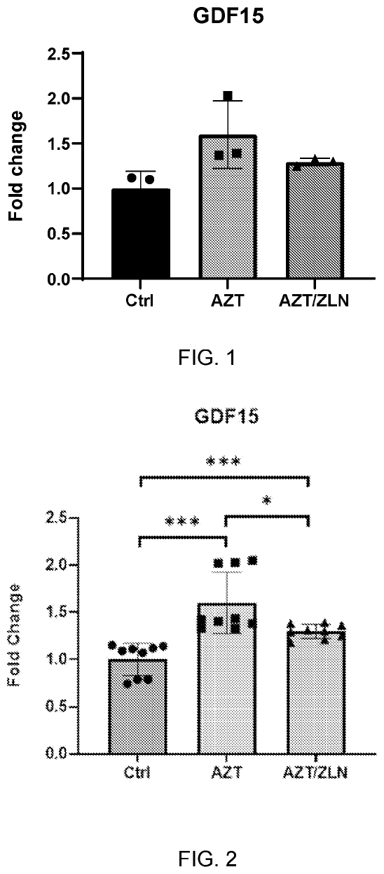

Results for two different sets of runs are shown in FIG. 1 and FIG. 2. As shown in both FIG. 1 and FIG. 2, cells treated with AZT show an increase in GDF-15 expression relative to NTC. Cells treated with AZT and ZLN-005 showed reduced GDF-15 expression compared to cells treated with AZT but not ZLN-005.

### 7. SPECIFIC EMBODIMENTS

The present disclosure is exemplified by the specific embodiments below.

1. A method for reducing plasma growth/differentiation factor 15 (GDF-15) in a subject, comprising administering to the subject an agent which is a compound of Formula (I):

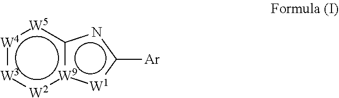

or a salt, hydrate, deuterated analog, or fluorinated analog thereof in an amount effective to reduce the subject's plasma GDF-15, wherein:
Ar is

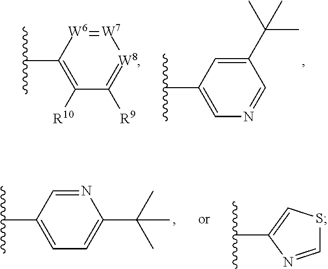

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁₋₄)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

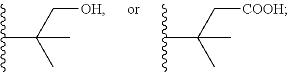

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

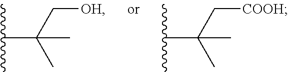

R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₅)alkoxycarbonyl, (C₁-C₆)alkoxycarbonylamino, methylthio, heterocyclyl, (C₁-C₁₀)oxaalkyl, CHR⁴NHR⁴⁵ and guanidine;
each of R⁴⁰ and R⁴¹ is independently hydrogen or (C₁-C₅) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₅)alkyl.

2. A method for reducing reductive stress in a subject in need thereof, comprising administering to the subject a compound of Formula (I):

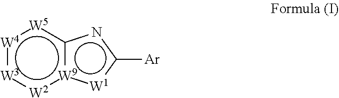

or a salt, hydrate, deuterated analog, or fluorinated analog thereof in an amount effective to reduce reductive stress in the subject, wherein:
Ar is

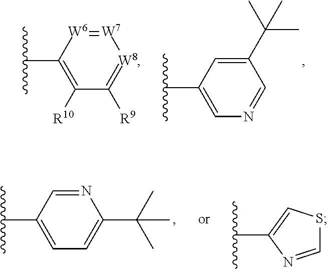

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁₋₄)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

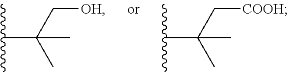

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

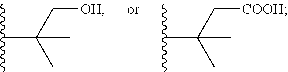

R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₅)alkoxycarbonyl, (C₁-C₅)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

3. A method for treating a subject having or at risk of developing a disease or dysfunction associated with reductive stress, comprising administering to the subject a compound of Formula (I):

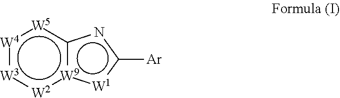

or a salt, hydrate, deuterated analog, or fluorinated analog thereof in an amount effective to reduce reductive stress in the subject, wherein:
Ar is

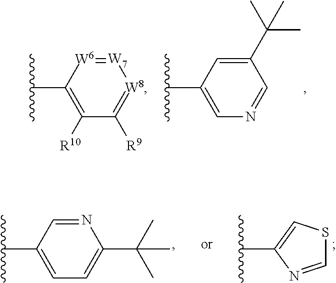

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, We may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁₋₄)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

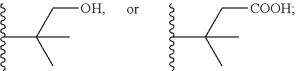

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

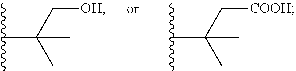

R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₆)alkoxycarbonyl, (C₁-C₅)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₅) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

4. The method of embodiment 2 or embodiment 3, wherein the reductive stress is NADH-reductive stress.

5. The method of any one of embodiments 1 to 4, wherein the subject has or is at risk of developing a disease or dysfunction associated with reductive stress.

6. The method of embodiment 5, wherein the disease or dysfunction associated with reductive stress is associated with an elevated NADH/NAD+ ratio in plasma and/or one or more tissues.

7. The method of embodiment 5 or embodiment 6, wherein the disease or dysfunction associated with reductive stress is associated with an elevated blood lactate/pyruvate ratio.

8. The method of any one of embodiments 5 to 7, wherein the disease or dysfunction associated with reductive stress is a mitochondrial disease or dysfunction, a cardiovascular disease or dysfunction, a liver disease or dysfunction, a lung disease or dysfunction, a kidney disease or dysfunction, or a cancer.

9 The method of embodiment 8, wherein the disease or dysfunction associated with reductive stress is a mitochondrial disease or dysfunction.

10. The method of method of embodiment 9, wherein the mitochondrial disease or dysfunction is a primary mitochondrial disease.

11. The method of embodiment 10, wherein the primary mitochondrial disease is mitochondrial encephalomyopathy lactic acidosis and strokelike episodes (MELAS).

12. The method of embodiment 10, wherein primary mitochondrial disease is Leigh syndrome.

13. The method of embodiment 10, wherein the primary mitochondrial disease is Kearns-Sayre syndrome.

14. The method of embodiment 10, wherein the primary mitochondrial disease is Alpers-Huttenlocher syndrome.

15. The method of embodiment 10, wherein the primary mitochondrial disease is Ataxia neuropathy syndrome.

16. The method of embodiment 9, wherein the mitochondrial disease or dysfunction is a secondary mitochondrial dysfunction.

17. The method of embodiment 16, wherein the secondary mitochondrial dysfunction is mitochondrial dysfunction secondary to diabetes, heart disease, cancer, a kidney disease, or a neurodegenerative disorder.

18. The method of embodiment 17, wherein the secondary mitochondrial dysfunction is mitochondrial dysfunction secondary to diabetes.

19. The method of embodiment 17, wherein the secondary mitochondrial dysfunction is mitochondrial dysfunction secondary to heart disease.

20. The method of embodiment 17, wherein the secondary mitochondrial dysfunction is mitochondrial dysfunction secondary to cancer.

21. The method of embodiment 17, wherein the secondary mitochondrial dysfunction is mitochondrial dysfunction secondary to a kidney disease.

22. The method of embodiment 17, wherein the secondary mitochondrial dysfunction is mitochondrial dysfunction secondary to a neurodegenerative disorder.

23. The method of embodiment 17, wherein the secondary mitochondrial dysfunction is mitochondrial dysfunction secondary to Friedreich's Ataxia.

24. The method of embodiment 17, wherein the secondary mitochondrial dysfunction is mitochondrial dysfunction secondary to Duchenne Muscular Dystrophy.

25. The method of embodiment 17, wherein the secondary mitochondrial dysfunction is mitochondrial dysfunction secondary to Becker Muscular Dystrophy.

26. The method of embodiment 8, wherein the disease or dysfunction associated with reductive stress is a cardiovascular disease or dysfunction.

27. The method of embodiment 26, wherein the cardiovascular disease or dysfunction is heart disease 28. The method of embodiment 26, wherein the cardiovascular disease or dysfunction is atherosclerosis.

29. The method of embodiment 8, wherein the disease or dysfunction associated with reductive stress is a liver disease or dysfunction.

30. The method of embodiment 29, wherein the liver disease or dysfunction is a chronic liver disease or dysfunction.

31. The method of embodiment 29, wherein the liver disease or dysfunction is an acute liver disease or dysfunction.

32. The method of embodiment 8, wherein the disease or dysfunction associated with reductive stress is a lung disease or dysfunction.

33. The method of embodiment 32, wherein the lung disease or dysfunction is a chronic lung disease or dysfunction.

34. The method of embodiment 32, wherein the lung disease or dysfunction is an acute lung disease or dysfunction.

35. The method of embodiment 8, wherein the disease or dysfunction associated with reductive stress is a kidney disease or dysfunction.

36. The method of embodiment 35, wherein the lung disease or dysfunction is a chronic kidney disease or dysfunction.

37. The method of embodiment 35, wherein the lung disease or dysfunction is an acute kidney disease or dysfunction.

38. The method of embodiment 8, wherein the disease or dysfunction associated with reductive stress is a cancer.

39. The method of embodiment 38, wherein the cancer is a solid tumor.

40. The method of embodiment 38, wherein the cancer is a hematologic cancer.

41. The method of any one of embodiments 5 to 40, wherein the subject has the disease or dysfunction associated with reductive stress.

42. The method of embodiment 41, which comprises administering an amount of the agent effective to ameliorate a symptom of the disease or dysfunction.

43. The method of any one of embodiments 5 to 40, wherein the subject is at risk of developing the disease or dysfunction associated with reductive stress.

44. The method of any one of embodiments 1 to 43, wherein the subject is receiving or has received treatment with one or more nucleoside analogs, optionally wherein the one or more nucleoside analogs comprise azidothymidine (AZT).

45. The method of any one of embodiments 1 to 44, wherein the subject has one or more biomarkers of reductive stress.

46. The method of embodiment 45, wherein the one or more biomarkers comprise an elevated NADH/NAD+ molar ratio in one or more cell types.

47. The method of embodiment 46, wherein the NADH/NAD+ molar ratio is elevated relative to a NADH/NAD+ reference value for the one or more cell types in a population of healthy subjects.

48. The method of any one of embodiments 45 to 47, wherein the one or more biomarkers comprise an elevated plasma GDF-15 level.

49. The method of embodiment 48, wherein the elevated plasma GDF-15 level is elevated relative to a GDF-15 reference value from a population of healthy subjects.

50. The method of embodiment 48, wherein the elevated plasma GDF-15 level is greater than 1000 ng/l.

51. The method of any one of embodiments 45 to 50, wherein the one or more biomarkers comprise an elevated tissue GDF-15 level.

52. The method of embodiment 51, wherein the elevated tissue GDF-15 level is elevated relative to a GDF-15 reference value from a population of healthy subjects.

53. The method of any one of embodiments 45 to 52, wherein the one or more biomarkers comprise an elevated plasma FGF-21 level.

54. The method of embodiment 53, wherein the elevated plasma FGF-21 level is elevated relative to a FGF-21 reference value from a population of healthy subjects.

55. The method of any one of embodiments 45 to 54, wherein the one or more biomarkers comprise an elevated tissue FGF-21 level.

56. The method of embodiment 55, wherein the elevated tissue FGF-21 level is elevated relative to a FGF-21 reference value from a population of healthy subjects.

57. The method of any one of embodiments 45 to 56, wherein the one or more biomarkers comprise an elevated plasma FGF-23 level.

58. The method of embodiment 57, wherein the elevated plasma FGF-23 level is elevated relative to a FGF-23 reference value from a population of healthy subjects.

59. The method of any one of embodiments 45 to 58, wherein the one or more biomarkers comprise an elevated tissue FGF-23 level.

60. The method of embodiment 59, wherein the elevated tissue FGF-23 level is elevated relative to a FGF-23 reference value from a population of healthy subjects.

61. The method of any one of embodiments 45 to 60, wherein the one or more biomarkers comprise an elevated blood lactate/pyruvate molar ratio.

62. The method of embodiment 61, wherein the elevated blood lactate/pyruvate ratio is elevated relative to a lactate/pyruvate molar ratio reference value from a population of healthy subjects.

63. The method of embodiment 61, wherein the subject has a blood lactate/pyruvate molar ratio of 20 to 35.

64. The method of embodiment 61, wherein the subject has a blood lactate/pyruvate molar ratio of 20 to 50.

65. The method of embodiment 61, wherein the subject has a blood lactate/pyruvate molar ratio of 30 to 50.

66. The method of embodiment 61, wherein the subject has a blood lactate/pyruvate molar ratio of 30 to 40.

67. The method of any one of embodiments 61 to 66, wherein the subject has a blood lactate/pyruvate molar ratio of at least 20.

68. The method of any one of embodiments 61 to 66, wherein the subject has a blood lactate/pyruvate molar ratio of at least 25.

69. The method of any one of embodiments 61 to 66, wherein the subject has a blood lactate/pyruvate molar ratio of at least 30.

70. The method of any one of embodiments 61 to 66, wherein the subject has a blood lactate/pyruvate molar ratio of at least 35.

71. The method of any one of embodiments 1 to 70, which comprises administering an amount of the agent effective to reduce one or more biomarkers of reductive stress in the subject.

72. The method of embodiment 71, which comprises administering an amount of the agent effective to reduce NADH/NAD+ molar ratio in one or more cell types of the subject.

73. The method of embodiment 71 or embodiment 72, which comprises administering an amount of the agent effective to reduce the subject's plasma GDF-15.

74. The method of any one of embodiments 71 to 73, which comprises administering an amount of the agent effective to reduce GDF-15 in a tissue of the subject.

75. The method of any one of embodiments 71 to 74, which comprises administering an amount of the agent effective to reduce the subject's plasma FGF-21.

76. The method of any one of embodiments 71 to 75, which comprises administering an amount of the agent effective to reduce FGF-21 in a tissue of the subject.

77. The method of any one of embodiments 71 to 76, which comprises administering an amount of the agent effective to reduce the subject's plasma FGF-23.

78. The method of any one of embodiments 71 to 77, which comprises administering an amount of the agent effective to reduce FGF-23 in the tissue of a subject.

79. The method of any one of embodiments 71 to 78, which comprises administering an amount of the agent effective to reduce the subject's blood lactate/pyruvate ratio.

80. The method of any one of embodiments 1 to 79, wherein the agent is

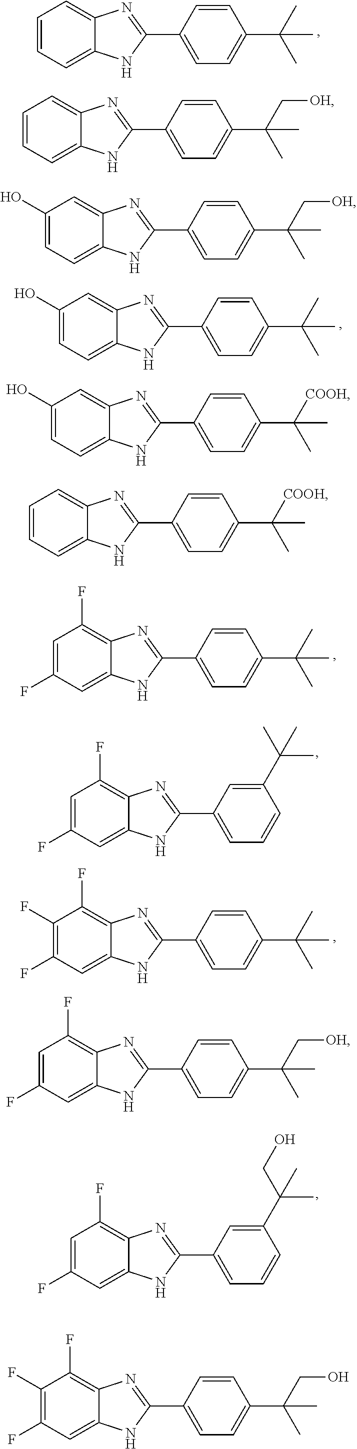

or a salt, hydrate, deuterated analog, or fluorinated analog thereof.

81. The method of embodiment 80, wherein the agent is

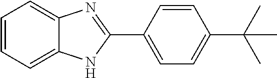 [SMILES: CC(C)(C)c1ccc(-c2nc3ccccc3[nH]2)cc1]

or a salt, hydrate, deuterated analog, or fluorinated analog thereof.

82. The method of embodiment 81, wherein the agent is

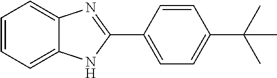 [SMILES: CC(C)(C)c1ccc(-c2nc3ccccc3[nH]2)cc1]

or a salt thereof.

83. The method of embodiment 80, wherein the agent is

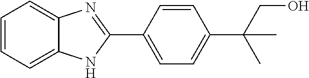 [SMILES: CC(C)(CO)c1ccc(-c2nc3ccccc3[nH]2)cc1]

or a salt, hydrate, deuterated analog, or fluorinated analog thereof.

84. The method of embodiment 80, wherein the agent is

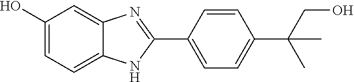 [SMILES: CC(C)(CO)c1ccc(-c2nc3cc(O)ccc3[nH]2)cc1]

or a salt, hydrate, deuterated analog, or fluorinated analog thereof.

85. The method of embodiment 80, wherein the agent is

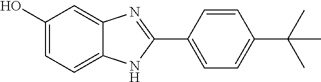 [SMILES: CC(C)(C)c1ccc(-c2nc3cc(O)ccc3[nH]2)cc1]

or a salt, hydrate, deuterated analog, or fluorinated analog thereof.

86. The method of embodiment 80, wherein the agent is

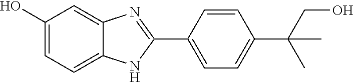 [SMILES: CC(C)(CO)c1ccc(-c2nc3cc(O)ccc3[nH]2)cc1]

or a salt, hydrate, deuterated analog, or fluorinated analog thereof.

87. The method of embodiment 80, wherein the agent is

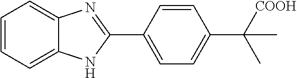 [SMILES: CC(C)(C(=O)O)c1ccc(-c2nc3ccccc3[nH]2)cc1]

or a salt, hydrate, deuterated analog, or fluorinated analog thereof.

88. The method of embodiment 80, wherein the agent is

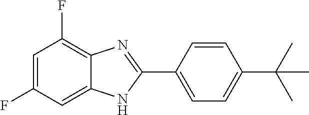 [SMILES: CC(C)(C)c1ccc(-c2nc3c(F)cc(F)cc3[nH]2)cc1]

or a salt, hydrate, deuterated analog, or fluorinated analog thereof.

89. The method of embodiment 80, wherein the agent is

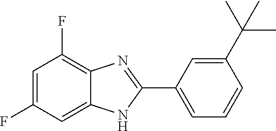 [SMILES: CC(C)(C)c1cccc(-c2nc3c(F)cc(F)cc3[nH]2)c1]

or a salt, hydrate, deuterated analog, or fluorinated analog thereof.

90. The method of embodiment 80, wherein the agent is

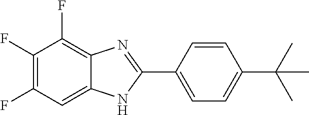 [SMILES: CC(C)(C)c1ccc(-c2nc3c(F)c(F)c(F)cc3[nH]2)cc1]

or a salt, hydrate, deuterated analog, or fluorinated analog thereof.

91. The method of embodiment 80, wherein the agent is

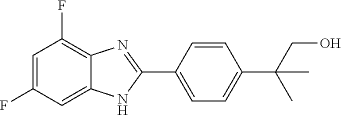 [SMILES: CC(C)(CO)c1ccc(-c2nc3c(F)cc(F)cc3[nH]2)cc1]

or a salt, hydrate, deuterated analog, or fluorinated analog thereof.

92. The method of embodiment 80, wherein the agent is

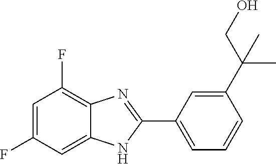 [SMILES: CC(C)(CO)c1cccc(-c2nc3c(F)cc(F)cc3[nH]2)c1]

or a salt, hydrate, deuterated analog, or fluorinated analog thereof.

93. The method of embodiment 80, wherein the agent is

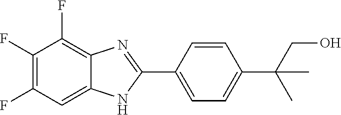 [SMILES: CC(C)(CO)c1ccc(-c2nc3c(F)c(F)c(F)cc3[nH]2)cc1]

or a salt, hydrate, deuterated analog, or fluorinated analog thereof.

94. The method of any one of embodiments 1 to 93, wherein the agent is administered enterically.

95. The method of embodiment 94, wherein the agent is administered by mouth.

96. The method of any one of embodiments 1 to 93, wherein the agent is administered by mouth (p.o.).

97. The method of any one of embodiments 1 to 96, wherein the subject has a neurodegenerative disorder.

98. The method of any one of embodiments 1 to 96, wherein the subject does not have a neurodegenerative disorder.

99. The method of any one of embodiments 1 to 98, wherein the subject does not have a systemic immune activation syndrome.

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method for reducing plasma growth/differentiation factor 15 (GDF-15) in a subject, comprising administering to the subject an agent which is a compound of Formula (I): 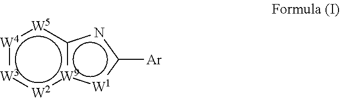
or a salt, hydrate, deuterated analog, or fluorinated analog thereof in an amount effective to reduce the subject's plasma GDF-15, wherein:
Ar is 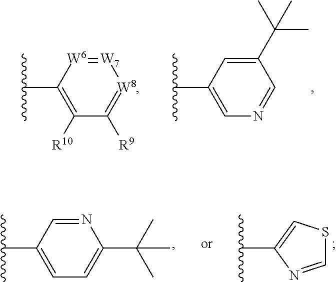
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁₋₄)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy, 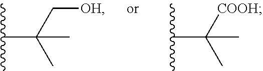
R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 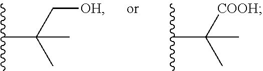
R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₅) hydrocarbyl:
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

2. A method for reducing reductive stress in a subject in need thereof, comprising administering to the subject a compound of Formula (I): 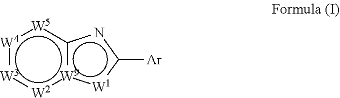
or a salt, hydrate, deuterated analog, or fluorinated analog thereof in an amount effective to reduce reductive stress in the subject, wherein:
Ar is 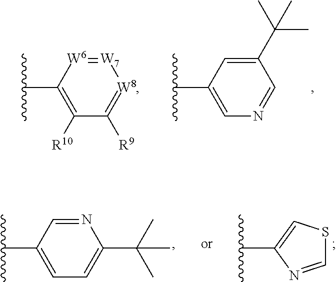
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁₋₄)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy, 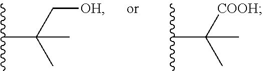
R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 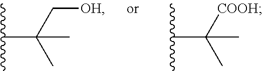
R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₆)alkoxycarbonyl, (C₁-C₅)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

3. A method for treating a subject having or at risk of developing a disease or dysfunction associated with reductive stress, comprising administering to the subject a compound of Formula (I): 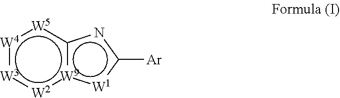
or a salt, hydrate, deuterated analog, or fluorinated analog thereof in an amount effective to reduce reductive stress in the subject, wherein:
Ar is 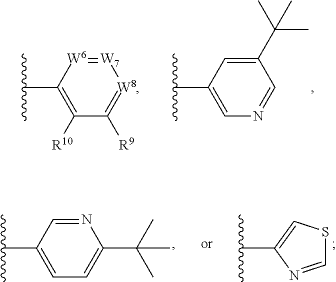
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro (C₁-C₄)alkyl, (C₁-C₄)alkyl, (C₁-C₄)alkoxy, perfluoro (C₁-C₄)alkoxy, (C₁-C₄) acyl, (C₁-4)alkoxy (C₁-C₄)alkyl, hydroxy (C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄) acylamino;
each of R⁶ and R¹⁰ is independently hydrogen, deuterium, halo, (C₁-C₃)alkyl, perfluoro (C₁-C₃)alkyl, hydroxy, (C₁-C₃)alkoxy, perfluoro (C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy, 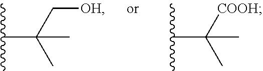
R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 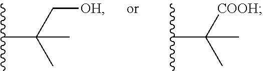
R³⁰ is (C₁-C₁₀) hydrocarbyl, (C₁-C₁₀) hydrocarbyl substituted with amino, (C₁-C₁₀) hydrocarbyl substituted with (C₁-C₄) hydrocarbyl, (C₁-C₁₀) hydrocarbyl substituted with carboxyl, carboxy, (C₁-C₅)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆) hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

4. The method of claim 2 or claim 3, wherein the reductive stress is NADH-reductive stress.

5. The method of any one of claims 1 to 4, wherein the subject has or is at risk of developing a disease or dysfunction associated with reductive stress.

6. The method of claim 5, wherein the disease or dysfunction associated with reductive stress is associated with an elevated NADH/NAD+ ratio in plasma and/or one or more tissues.

7. The method of claim 5 or claim 6, wherein the disease or dysfunction associated with reductive stress is associated with an elevated blood lactate/pyruvate ratio.

8. The method of any one of claims 5 to 7, wherein the disease or dysfunction associated with reductive stress is a mitochondrial disease or dysfunction, a cardiovascular disease or dysfunction, a liver disease or dysfunction, a lung disease or dysfunction, a kidney disease or dysfunction, or a cancer.

9. The method of claim 8, wherein the disease or dysfunction associated with reductive stress is a mitochondrial disease or dysfunction.

10. The method of method of claim 9, wherein the mitochondrial disease or dysfunction is a primary mitochondrial disease.

11. The method of claim 10, wherein the primary mitochondrial disease is mitochondrial encephalomyopathy lactic acidosis and strokelike episodes (MELAS), Leigh syndrome, Kearns-Sayre syndrome, Alpers-Huttenlocher syndrome, or Ataxia neuropathy syndrome.

12. The method of claim 9, wherein the mitochondrial disease or dysfunction is a secondary mitochondrial dysfunction.

13. The method of claim 12, wherein the secondary mitochondrial dysfunction is mitochondrial dysfunction secondary to diabetes, heart disease, cancer, a kidney disease, or a neurodegenerative disorder.

14. The method of claim 12, wherein the secondary mitochondrial dysfunction is mitochondrial dysfunction secondary to Friedreich's Ataxia, Duchenne Muscular Dystrophy, or Becker Muscular Dystrophy.

15. The method of claim 8, wherein the disease or dysfunction associated with reductive stress is a cardiovascular disease or dysfunction.

16. The method of claim 15, wherein the cardiovascular disease or dysfunction is heart disease or atherosclerosis.

17. The method of claim 8, wherein the disease or dysfunction associated with reductive stress is a liver disease or dysfunction.

18. The method of claim 17, wherein the liver disease or dysfunction is a chronic liver disease or dysfunction.

19. The method of claim 17, wherein the liver disease or dysfunction is an acute liver disease or dysfunction.

20. The method of claim 8, wherein the disease or dysfunction associated with reductive stress is a lung disease or dysfunction.

21. The method of claim 20, wherein the lung disease or dysfunction is a chronic lung disease or dysfunction.

22. The method of claim 20, wherein the lung disease or dysfunction is an acute lung disease or dysfunction.

23. The method of claim 8, wherein the disease or dysfunction associated with reductive stress is a kidney disease or dysfunction.

24. The method of claim 23, wherein the lung disease or dysfunction is a chronic kidney disease or dysfunction.

25. The method of claim 23, wherein the lung disease or dysfunction is an acute kidney disease or dysfunction.

26. The method of claim 8, wherein the disease or dysfunction associated with reductive stress is a cancer.

27. The method of claim 26, wherein the cancer is a solid tumor.

28. The method of claim 26, wherein the cancer is a hematologic cancer.

29. The method of any one of claims 5 to 28, wherein the subject has the disease or dysfunction associated with reductive stress.

30. The method of claim 29, which comprises administering an amount of the agent effective to ameliorate a symptom of the disease or dysfunction.

31. The method of any one of claims 5 to 28, wherein the subject is at risk of developing the disease or dysfunction associated with reductive stress.

32. The method of any one of claims 1 to 31, wherein the subject is receiving or has received treatment with one or more nucleoside analogs, optionally wherein the one or more nucleoside analogs comprise azidothymidine (AZT).

33. The method of any one of claims 1 to 32, wherein the subject has one or more biomarkers of reductive stress.

34. The method of claim 33, wherein the one or more biomarkers comprise an elevated NADH/NAD+ molar ratio in one or more cell types.

35. The method of claim 34, wherein the NADH/NAD+ molar ratio is elevated relative to a NADH/NAD+ reference value for the one or more cell types in a population of healthy subjects.

36. The method of any one of claims 33 to 35, wherein the one or more biomarkers comprise an elevated plasma GDF-15 level, optionally wherein the elevated plasma GDF-15 level is elevated relative to a GDF-15 reference value from a population of healthy subjects.

37. The method of claim 36, wherein the elevated plasma GDF-15 level is greater than 1000 ng/l.

38. The method of any one of claims 33 to 37, wherein the one or more biomarkers comprise an elevated tissue GDF-15 level, optionally wherein the elevated tissue GDF-15 level is elevated relative to a GDF-15 reference value from a population of healthy subjects.

39. The method of any one of claims 33 to 38, wherein the one or more biomarkers comprise an elevated plasma FGF-21 level, optionally wherein the elevated plasma FGF-21 level is elevated relative to a FGF-21 reference value from a population of healthy subjects.

40. The method of any one of claims 33 to 39, wherein the one or more biomarkers comprise an elevated tissue FGF-21 level, optionally wherein the elevated tissue FGF-21 level is elevated relative to a FGF-21 reference value from a population of healthy subjects.

41. The method of any one of claims 33 to 40, wherein the one or more biomarkers comprise an elevated plasma FGF-23 level, optionally wherein the elevated plasma FGF-23 level is elevated relative to a FGF-23 reference value from a population of healthy subjects.

42. The method of any one of claims 33 to 41, wherein the one or more biomarkers comprise an elevated tissue FGF-23 level, optionally wherein the elevated tissue FGF-23 level is elevated relative to a FGF-23 reference value from a population of healthy subjects.

43. The method of any one of claims 33 to 42, wherein the one or more biomarkers comprise an elevated blood lactate/pyruvate molar ratio.

44. The method of claim 43, wherein the elevated blood lactate/pyruvate ratio is elevated relative to a lactate/pyruvate molar ratio reference value from a population of healthy subjects.

45. The method of claim 43, wherein the subject has a blood lactate/pyruvate molar ratio of 20 to 35, 20 to 50, 30 to 50, or 30 to 40.

46. The method of any one of claims 43 to 45, wherein the subject has a blood lactate/pyruvate molar ratio of at least 20, at least 25, at least 30, or at least 35.

47. The method of any one of claims 1 to 46, which comprises administering an amount of the agent effective to reduce one or more biomarkers of reductive stress in the subject.

48. The method of claim 47, which comprises administering an amount of the agent effective to reduce NADH/NAD+ molar ratio in one or more cell types of the subject.

49. The method of claim 47 or claim 48, which comprises administering an amount of the agent effective to reduce the subject's plasma GDF-15.

50. The method of any one of claims 47 to 49, which comprises administering an amount of the agent effective to reduce GDF-15 in a tissue of the subject.

51. The method of any one of claims 47 to 50, which comprises administering an amount of the agent effective to reduce the subject's plasma FGF-21.

52. The method of any one of claims 47 to 51, which comprises administering an amount of the agent effective to reduce FGF-21 in a tissue of the subject.

53. The method of any one of claims 47 to 52, which comprises administering an amount of the agent effective to reduce the subject's plasma FGF-23.

54. The method of any one of claims 47 to 53, which comprises administering an amount of the agent effective to reduce FGF-23 in the tissue of a subject.

55. The method of any one of claims 47 to 54, which comprises administering an amount of the agent effective to reduce the subject's blood lactate/pyruvate ratio.

56. The method of any one of claims 1 to 55, wherein the agent is 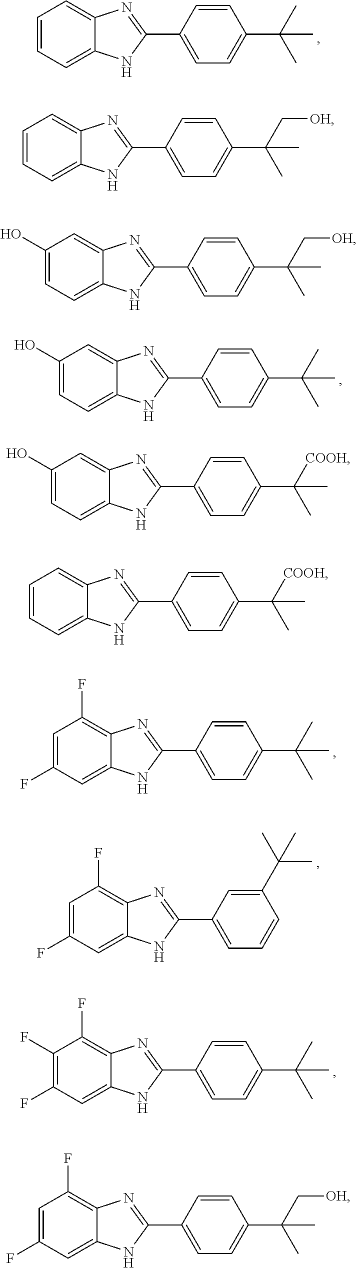 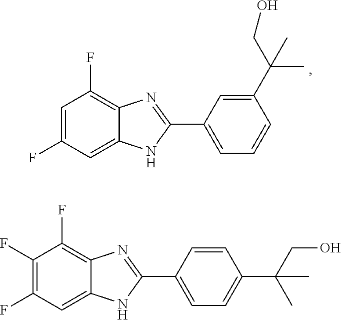 [SMILES: CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]
or a salt, hydrate, deuterated analog, or fluorinated analog thereof.

57. The method of claim 56, wherein the agent is 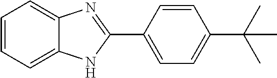 [SMILES: CC(C)(C)c1ccc(-c2nc3ccccc3[nH]2)cc1]
or a salt, hydrate, deuterated analog, or fluorinated analog thereof.

58. The method of any one of claims 1 to 57, wherein the agent is administered enterically, optionally by mouth.

59. The method of any one of claims 1 to 58, wherein the subject has a neurodegenerative disorder.

60. The method of any one of claims 1 to 58, wherein the subject does not have a neurodegenerative disorder.

61. The method of any one of claims 1 to 60, wherein the subject does not have a systemic immune activation syndrome.
